(19) United States
(12) Patent Application Publication
(10) Pub. No.: US 2010/0286469 A1
(43) Pub. Date: Nov. 11, 2010

(54) Device for attenuating cellular metabolism

(76) Inventor: Ivan Rampl, Brno (CZ)

Correspondence Address: SMITH, GAMBRELL & RUSSELL, 1130 CONNECTICUT AVENUE, N.W., SUITE 1130, WASHINGTON, DC, 20036, US

(21) Appl. No.: 12/308,396

(22) PCT Filed: Jun. 14, 2007
(86) PCT No.: PCT/CZ2007/000051

§ 371 Date: Jul. 29, 2010

(30) Foreign Application Priority Data
Jun. 14, 2006 (CZ) PV 2006-379

(51) Int. Cl. A61N 2/04

(52) U.S. Cl. 600/14

(57) ABSTRACT

The invention concerns a device for attenuating the metabolism of cells, which is formed by a computer (1)-controlled generator (2) of pulse signal, connected to an applicator (3), which is provided with at least one toroidal coil (4) or a concentrically arranged assembly (5) of at least two parallel-connected toroidal coils (4), whose leads are connected to the output of pulse signal generator (2). The pulse signal generator (2) is either a generator of unipolar rectangular signals with very steep leading and trailing edges or a generator of bipolar rectangular signals with a high rate of bipolar signal sweep. The toroidal coils (4) or concentrically arranged assemblies (5) of at least two parallel-connected toroidal coils (4) can be positioned on a surface, for example planar or spherical, and can have spatially adjustable symmetry axes for directing the pulse vector magnetic potential. The principle of the device function consists in generating a pulse vector magnetic potential, which has a destroying and irreversible effect on live material. At present it has been experimentally verified that the pulse vector magnetic potential has a pronounced effect on electrochemical processes, for the time being in selected types of live cells, even if the action of magnetic, electric and electromagnetic fields is eliminated.

### BACKGROUND OF THE INVENTION

The invention concerns a device for attenuating the metabolism of cells, which is formed by a computer-controlled generator of pulsed signal, connected to an applicator.

### PRIOR ART

One of the most pernicious and most widespread diseases that people today suffer from is cancer, a malign tumorous growth of cells in a human body.

Today, medical science has three fundamentally different procedures for combating this growth; they can be applied either separately or in mutual combination.

The first procedure is chemotherapy, which is administered to the patient either orally or intravenously. A disadvantage of chemotherapy is that the chemical substances administered cannot be targeted at only the cells that are detrimental to the organism; chemotherapy can result in not only destroyed tumorous cells but also greatly damaged cells that are beneficial for the human body. The patient can thus sometimes be saved but often at the cost of impairing his/her health.

Another procedure consists in removing the malign tumour by operation. A disadvantage of this procedure is that its success depends on whether or not the whole tumour is removed with a margin of surrounding normal tissue and whether or not further metastases infiltrate into the body. Moreover, any intervention via operation will weaken the patient's body, which has already been weakened by the malign tumour. Thus only a person who, in spite of the disease, is strong enough to survive such treatment can be operated on.

The third, widely practised procedure consists in irradiating the malign tumour with different types of radiation, from X-ray radiation to gamma radiation. A disadvantage of this procedure is that these rays destroy all the tissue that stands in their way and it is difficult to focus them on the spots only that require irradiation.

### SUBJECT MATTER OF THE INVENTION

The fact that there does not exist a device that makes use of the attenuating effect of pulse vector magnetic potential on the metabolism of cells is solved by a device for attenuating cellular metabolism that consists of a computer-controlled generator of pulse signal connected to an applicator, in which the essence of the invention is that the applicator is provided with at least one toroidal coil for producing a pulse vector magnetic potential, the toroidal coil being connected to the output of pulse signal generator. In a preferred embodiment, however, the applicator can also be provided with a concentrically arranged assembly of at least two parallel-connected toroidal coils whose leads are connected to the output of pulse signal generator. The pulse signal generator is either a generator of unipolar rectangular signals with very steep leading and trailing edges or a generator of bipolar rectangular signals with a high sweep rate of bipolar signal.

In another preferred embodiment, the applicator is formed by at least two adjacent toroidal coils positioned on a surface, with the coil leads connected to the output of pulse signal generator. The preferred surface on which the toroidal coils are positioned is spherical.

In yet another preferred embodiment, the applicator is formed by at least two adjacent concentric assemblies of toroidal coils connected in parallel and positioned on a surface, whose leads are connected to the output of pulse signal generator. The preferred surface on which the assemblies of parallel-connected toroidal coils are positioned is spherical.

In a further preferred embodiment of the device of the invention, the applicator is formed by at least two toroidal coils positioned on the circumference of a circle, with spatially adjustable symmetry axes pointing to the focus, with the coil leads connected to the output of pulse signal generator.

In another preferred embodiment of the device of the invention, the applicator is formed by at least two adjacent parallel-connected toroidal coils assembled concentrically on the circumference of a circle, with spatially adjustable symmetry axes, whose leads are connected to the output of pulse signal generator.

In a preferred embodiment of this invention, the toroidal coils are of insulated conductor and placed in metallic or non-metallic casings.

In another preferred embodiment, the toroidal coils are of superconductive material and placed in thermostatic protective casings in order to maintain superconductive conditions.

### BRIEF DESCRIPTIONS OF THE DRAWINGS

Below, the invention will be described in detail according to the drawings attached, where

FIG. 1 gives the schematic block diagram of the connection of the device of this invention;

FIG. 2 illustrates the applicator with one toroidal coil,

FIG. 3 illustrates the applicator with a number of toroidal coils positioned on a surface,

FIG. 4 illustrates the applicator with a concentrically positioned assembly of several parallel-connected toroidal coils,

FIG. 5 illustrates the applicator with a number of simple toroidal coils or concentrically positioned parallel-connected toroidal coils located on a part of spherical surface,

FIG. 6 illustrates the applicator with an assembly formed by a number of simple toroidal coils or concentrically positioned parallel-connected toroidal coils with spatially adjustable symmetry axes and located on the circumference of a circle.

### DESCRIPTION OF PREFERRED EMBODIMENTS OF THE INVENTION

When examining whether it would be possible to substitute existing kinds of radiation that are used to destroy malign tumours and stop their growth by some other kind of force acting on malignantly growing cells, the author of this invention employed toroidal coils and the pulse vector magnetic potential generated by them.

The vector magnetic potential was initially defined as a mathematical aid in the description of magnetic field relative to the description of dual electrical field, and its real existence was not assumed, in contrast to the scalar electric potential. In the latter half of the twentieth century, however, the view of this physical phenomenon was changed following the discovery of its action of force.

With the exception of some experiments made worldwide with the aim of proving by physical methods the real existence of static or variable vector magnetic potential, whose results can be disputed, there exists up to now just one, many times verified measurable effect. Via a purely physical experiment, carried out by Aharonov and Bohm (1956), the real effect of the field of vector magnetic potential was discovered and proved on the quantum mechanics level. Their experiment resulted in establishing the changes in the momentum of electrons travelling in a vacuum due to this field.

A significant property of the vector magnetic potential is its presence in a space separated and insulated from the space of the magnetic field that led to the existence of this potential. As a direct consequence of this specific phenomenon, a hypothesis appeared claiming that the vector magnetic potential is probably the fundamental mechanism of gravitation and ranks with fundamental physical forces. However, no practically utilizable applications of this physical quantity have up to now been recorded and nobody has succeeded in detecting or measuring its effects on live matter either.

Surprisingly enough, it has been established and experimentally verified that the pulse vector magnetic potential has a pronounced negative effect on electrochemical processes taking place in selected types of live cell even if the action of magnetic, electric and electromagnetic fields is eliminated.

Based on the above knowledge, devices have been designed whose activities consist, by this invention, in the destroying and irreversible effect of pulse vector magnetic potential on live material. By developing the fundamental principle of the device different design variants of the device can be obtained.

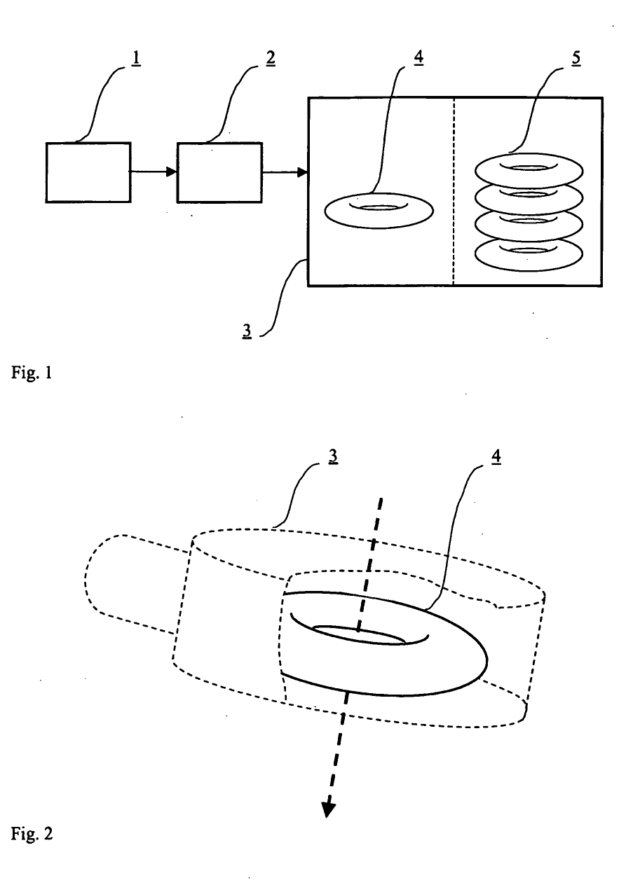

The schematic block diagram of the connection of the device for attenuating the metabolism of cells by the action of pulse vector magnetic potential in FIG. 1 consists of computer 1-controlled generator 2 of pulse signal and an applicator 3 for generating a pulse vector magnetic potential, the basic element of applicator 3 being a toroidal coil 4 of insulated conductor or an assembly 5 of concentrically positioned toroidal coils 4.

The design variant of applicator 3 with toroidal coil 4 for local therapy of the skin and hypodermis or mucosa is shown in FIG. 2, where the dashed axis of toroidal coil 4 simultaneously gives the direction the vector magnetic potential action on live matter.

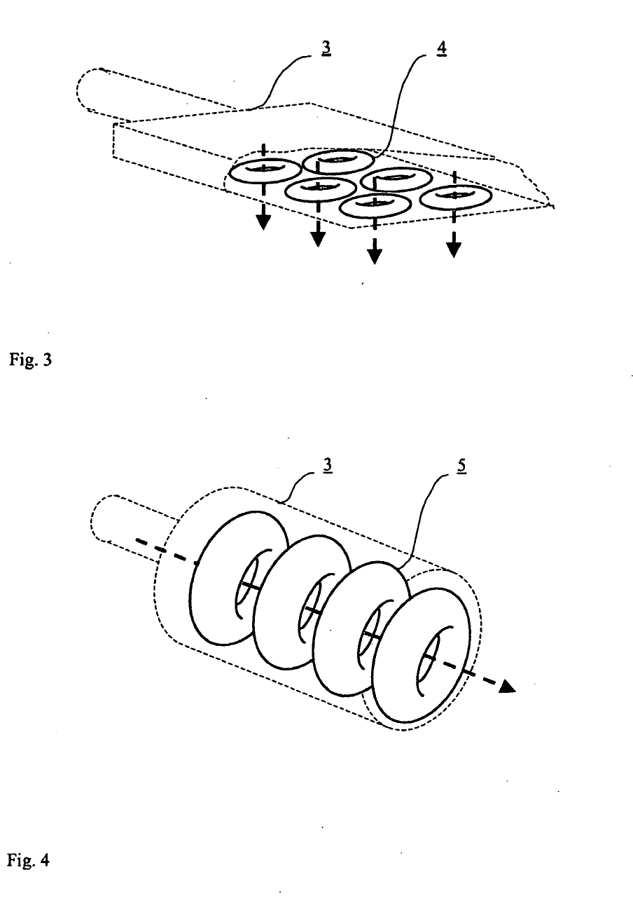

The design variant of applicator 3 with a number of surface-wise loeated toroidal coils 4 for surface therapy of parts of the skin and hypodermis or mucosa is illustrated in FIG. 3.

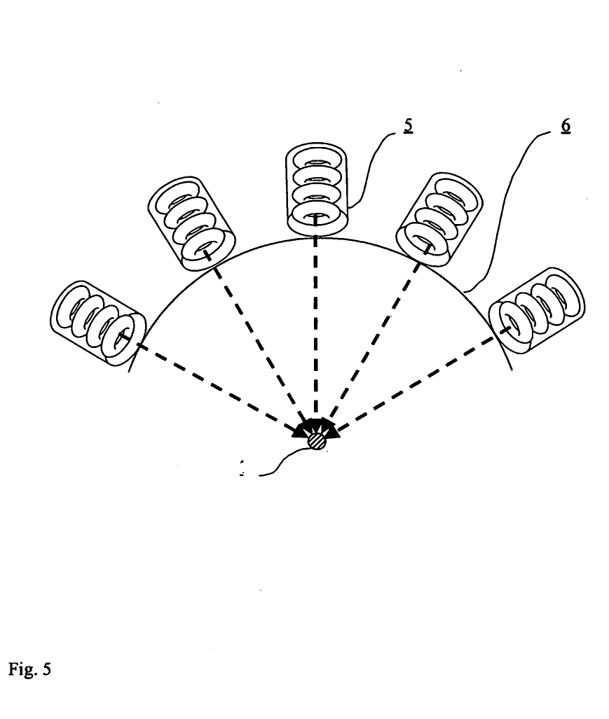

The design variant of applicator 3 with assembly 5 of concentrically positioned toroidal coils 4 for local therapy of deeper layers below the skin, with concentrated action, is illustrated in FIG. 4.

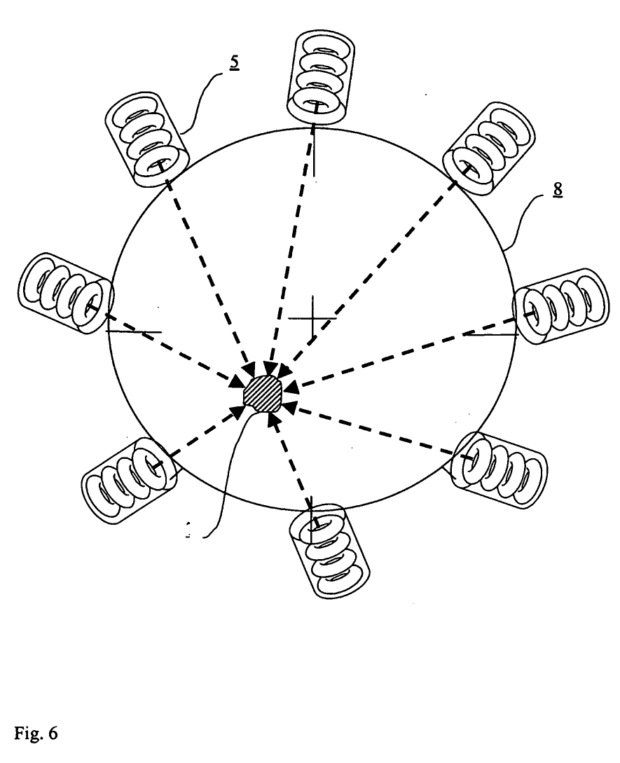

The design variant of applicator 3 with a number of assemblies 5 of concentrically positioned toroidal coils 4 located on the surface of a part of spherical area 6 with dashed-line symmetry axes pointing to the focus 7 is illustrated in FIG. 5, with applicator 3 being designed for local therapy of deeper layers below the skin, with limited damage to the cells outside the site of maximum destruction.

The design variant of applicator 3 with an assembly formed by a number of assemblies 5 of toroidal coils 4 with spatially adjustable symmetry axes pointing to the focus 7 and positioned on the circumference of circle 8 is illustrated in FIG. 6, with toroidal coils 4 being supplied with pulse signals of pre-set parameters and phase differences such that the effect of pulse vector magnetic potential can be modified. Applicator 3 is designed for the local therapy of the deeper layers below the skin, with limited damage to the cells outside the site treated.

The software of computer 1 of controlled generator 2 enables controlling all the important parameters of the driving signals coming to individual toroidal coils 4 according to the therapeutic prescription and according to the response of the organism being treated. Depending on the type of applicator 3, the size and type of toroidal coils 4 or assemblies 5 of toroidal coils 4, and the size, frequency and pulse shape of the driving signal the attenuating effect can be directed to areas of different sizes and to different depths of live tissue.

Applicator 3 formed by assembly 5 of toroidal coils 4 amplifies the attenuating effect by enhancing the directional nature of applicator 3, with the attenuating effect being maximal in the symmetry axis of toroidal coil 4.

Applicator 3 can have toroidal coils 4 or assemblies 5 of toroidal coils 4 enclosed in metallic or non-metallic protective casings since the effect of the field of pulse vector magnetic potential penetrates through any metallic or non-metallic material.

Toroidal coils 4 used either separately or in assemblies 5 of toroidal coils 4 can be wound of superconducting material and held in casings that maintain superconductive conditions. This will enable using larger driving currents and, in particular, accelerating their time variation in toroidal coils 4, which provides the possibility of increasing the effect of pulse vector magnetic potential of the device and thus also increasing the effect of the device.

An advantage of the arrangement described is that toroidal coils 4 or assemblies 5 of toroidal coils 4 positioned on a spherical surface have the maximum attenuating effect in the focus of their symmetry axes while along the paths of the axes of individual drivers the live tissue is stressed less negatively.

If applicator 3 is formed by toroidal coils 4 or assemblies 5 of toroidal coils 4 with spatially adjustable symmetry axes and positioned on the circumference of a circle around live tissue, then the size, shape, duration, repetition frequency, and time shift of individual driving pulses enable concentrating the maximum attenuating effect into a determined focus of the symmetry axes of toroidal coils.

### INDUSTRIAL APPLICABILITY OF THE INVENTION

The results of present-day research into the contactless action of magnetic and light fields on live tissue combined with chemotherapy are applied with advantage and used medically particularly in the presence of a field of pulse vector magnetic potential. This enables, for example, programmed stimulation or, on the contrary, attenuation of cellular metabolism on large surfaces of skin or mucosa. Applying the action of force of pulse vector magnetic potential is of particular advantage in such therapeutic procedures where the human organism is, on method-of-treatment principles, exposed to intensive action of electromagnetic fields (in the spectral region of light, X-ray and ionization radiations) that damage the organism.

Given as an example can be on the one hand the device for stopping malignant growth on the surface of skin, hypodermis or mucosa, using the applicators according to FIG. 2, FIG. 3, FIG. 4 or FIG. 5, on the other hand the device for stopping malignant growth inside the organism, using the applicators according to FIG. 5 or FIG. 6.

1. A device for attenuating a cellular metabolism, comprising: a computer (1), controlled generator (2) of pulse signal connected to an applicator (3), wherein the applicator (3) is provided with at least one toroidal coil (4) of isolated conductor for producing a pulse vector magnetic potential, the toroidal coil (4) being connected to an output of a pulse signal generator (2).

2. The device of claim 1, characterized in that the applicator (3) is provided with a concentrically arranged assembly (5) of at least two parallelly-connected toroidal coils (4), the leads of which are connected to the output of the pulse signal generator (2).

3. The device of claim 1, characterised in that the pulse signal generator (2) is a generator of unipolar rectangular signals with very steep leading and trailing edges.

4. The device of claim 1, characterised in that the pulse signal generator (2) is a generator of bipolar rectangular signals with a high rate of bipolar signal sweep.

5. The device of claim 1, characterised in that the applicator (3) is formed by at least two adjacent toroidal coils (4) positioned on a surface, the leads of which are connected to the output of the pulse signal generator (2).

6. The device of claim 5, characterised in that the surface on which toroidal coils (4) are positioned is spherical.

7. The device of claim 2, characterised in that the applicator (3) is formed by at least two adjacent concentrically positioned surface-mounted assemblies (5) of parallelly-connected toroidal coils (4), the leads of which are connected to the output of the pulse signal generator (2).

8. The device of claim 7, characterised in that the surface on which the assemblies (5) of parallelly-connected toroidal coils (4) are positioned is spherical.

9. The device of claim 1, characterised in that the applicator (3) is formed by at least two toroidal coils (4) deployed on the circumference of a circle (8) with spatially adjustable symmetry axes pointing to the focus (7).

10. The device of claim 2, characterised in that the applicator (3) is formed by at least two adjacent, on the circumference of the circle (8) positioned and concentrically arranged assemblies (5) of parallelly-connected toroidal coils (4) with spatially adjustable symmetry axes, whose leads are connected to the output of the pulse signal generator (2).

11. The device of claim 1, characterised in that the toroidal coils (4) are of insulated conductor and are held in metallic or non-metallic protective casings.

12. The device of claim 1, characterised in that toroidal coils (4) are made of superconductive material and are held in thermostatic protective casings in order to maintain superconductive conditions.

13. The device of claim 2, characterised in that the pulse signal generator (2) is a generator of unipolar rectangular signals with very steep leading and trailing edges.

14. The device of claim 2, characterised in that the pulse signal generator (2) is a generator of bipolar rectangular signals with a high rate of bipolar signal sweep.

15. The device of claim 2, characterised in that the toroidal coils (4) are of insulated conductor and are held in metallic or non-metallic protective casings.

16. The device of claim 2, characterised in that toroidal coils (4) are made of superconductive material and are held in thermostatic protective casings in order to maintain superconductive conditions.
